(19) United States
(12) Patent Application Publication
(10) Pub. No.: US 2025/0191753 A1
(43) Pub. Date: Jun. 12, 2025

(54) Multi-Modal Insomnia Detection Using a Wearable Device

(71) Applicant: Samsung Electronics Co., Ltd., Suwon-si (KR)

(72) Inventors: Veronica Iwummadu, Tracy, CA (US); Dhwanil Damania, Danville, CA (US); Christel Taylor, Cupertino, CA (US); Sahil Pujara, San Mateo, CA (US); Matthew Wiggins, San Jose, CA (US)

(21) Appl. No.: 18/796,590

(22) Filed: Aug. 7, 2024

Related U.S. Application Data
(60) Provisional application No. 63/607,501, filed on Dec. 7, 2023.

(51) Int. Cl. G16H 50/20; A61B 5/00; A61B 5/01; A61B 5/024; A61B 5/0531; A61B 5/11; A61B 5/369

(52) U.S. Cl. CPC G16H 50/20; A61B 5/01; A61B 5/024; A61B 5/0531; A61B 5/11; A61B 5/369; A61B 5/681

(57) ABSTRACT

In one embodiment, a method includes detecting, by each of multiple sensors of a wearable device worn by a user, corresponding insomnia-related signals during at least a predetermined duration; determining, for each of the insomnia-related signals, a set of insomnia-indicating signals by comparing each insomnia-related signal to a corresponding threshold; and determining, by a trained machine-learning model and based on the determined sets of insomnia-indicating signals, an insomnia condition of the user.

### PRIORITY CLAIM

This application claims the benefit under 35 U.S.C. § 119 of U.S. Provisional Patent Application No. 63/607,501 filed Dec. 7, 2023, which is incorporated by reference herein.

### TECHNICAL FIELD

This application generally relates to multi-modal insomnia detection using a wearable device.

### BACKGROUND

Insomnia is a relatively common condition that disrupts a person's normal sleep-wake cycle, making it harder for the person to fall asleep, to stay asleep, and/or to get restorative sleep. Insomnia can have adverse effects on a person's health and overall well-being and is also linked to higher healthcare utilization and costs, especially in patients with coexisting medical or psychiatric disorders. For example, studies suggest that a large number of heart-disease conditions (e.g., atrial fibrillation) occur during insomnia sleep disorders.

Insomnia can include acute forms, which can include short-term disruptions in a person's ability to fall asleep or stay asleep. For example, a temporary stressor in a person's life may temporarily affect the person's sleep. Insomnia can also include chronic forms, which are longer-term disruptions to a person's sleep, and acute forms of insomnia may turn into chronic forms. For example, a person who has evidenced sleep disruptions for longer than three months may be considered to have chronic insomnia.

### BRIEF DESCRIPTION OF THE DRAWINGS

FIG. 1 illustrates an example method of determining an insomnia condition of a user using a wearable device that has multiple sensors that each collects information related to insomnia.

FIG. 2 illustrates an example architecture for determining an insomnia condition of a user.

FIG. 3 illustrates an example computing system.

### DESCRIPTION OF EXAMPLE EMBODIMENTS

Potential causes or indicators of insomnia can include predisposing factors, such as genetic factors or personality traits leading to physiological and cognitive hyperarousal. Other causes or indicators include precipitating factors, such as triggers involving grief or other environment-induced stressors. Other causes or indicators can include perpetuation factors, which are factors that allow insomnia to continue, even after a trigger or underlying condition is removed. For example, poor sleep hygiene (e.g., a poor sleeping environment with respect to light or noise) may perpetuate insomnia. At times, more than one underlying cause or factor may be present in a particular person's insomnia.

Molecular factors responsible for sleep-wake regulation include wake-promoting chemicals such as orexin, catecholamine, and histamine and sleep promoting chemicals such as GABA, serotonin, adenosine, melatonin, and prostaglandin D2. The orexin-mediated increased neuronal firing in the wake-promoting area and inhibition of the sleep-promoting area (ventrolateral preoptic nucleus and median preoptic nucleus) is one of the possible mechanisms contributing to insomnia, and may be known as the sleep switch model.

Physiological factors indicating, or potentially contributing to, insomnia include increased EEG fast frequencies during sleep, increased number of arousals during REM sleep, increased daytime sleep-onset latency, and short sleep duration. Other physiological factors include increased activity of the pituitary-adrenal axis, increased heart rate, altered heart-rate variability, increased metabolic rate, and increased body temperature.

Other factors affecting sleep include anxiety (such as anxiety over life events or anxiety over one's ability to fall asleep), depression, sadness, and other mental and physical factors. A full clinical diagnosis of insomnia involves determining whether, and to what extent, these health issues may be present, and determining the most likely underlying causes(es) of sleep disorders.

Current insomnia diagnoses typically require a clinical visit where an overnight sleep study using polysomnography is administered to a patient. After the sleep study, the user then be asked to create, over several weeks, a self-tracked diary of insomnia-related factors, such as caffeine intake, alcohol consumption, when the patient went to bed and woke up, and whether they observed any disruptions to their sleep. The user may also wear an actigraphy device, which uses an accelerometer to infer activity-rest cycles of the use. Upon completion of the diary and actigraphy stage (e.g., 3-4 weeks), the patient may be proscribed treatment. However, this approach to diagnosing insomnia requires a clinical visit, which is disruptive and time-consuming for patients. Moreover, people must first recognize symptoms of a sleep disorder and infer that the symptoms relate to a sleep disorder in order to set up a clinical visit in the first place. Therefore, this approach requires some level of self-detection and self-diagnosis on the part of a patient to start the clinical process. In addition, portions of the clinical process (e.g., the diary) require subjective determinations on behalf of the user, including determinations regarding their perceptions of their own sleep, which can be difficult for users to ascertain, as many sleep-related symptoms are not consciously recognized or remembered when they occur.

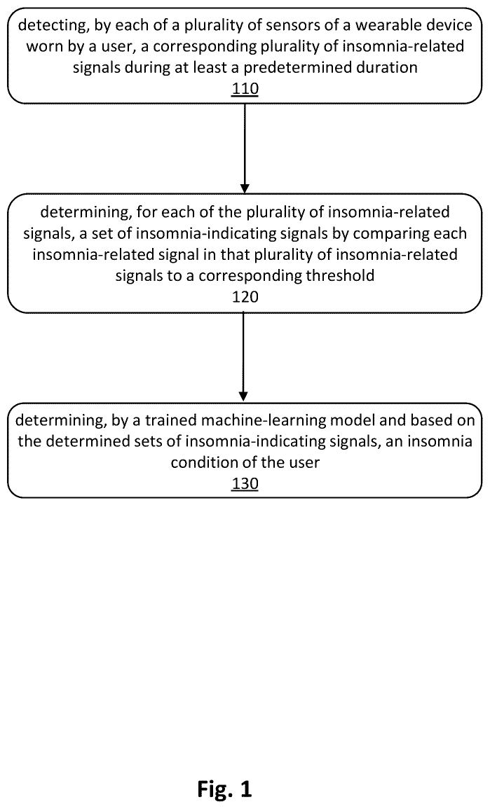

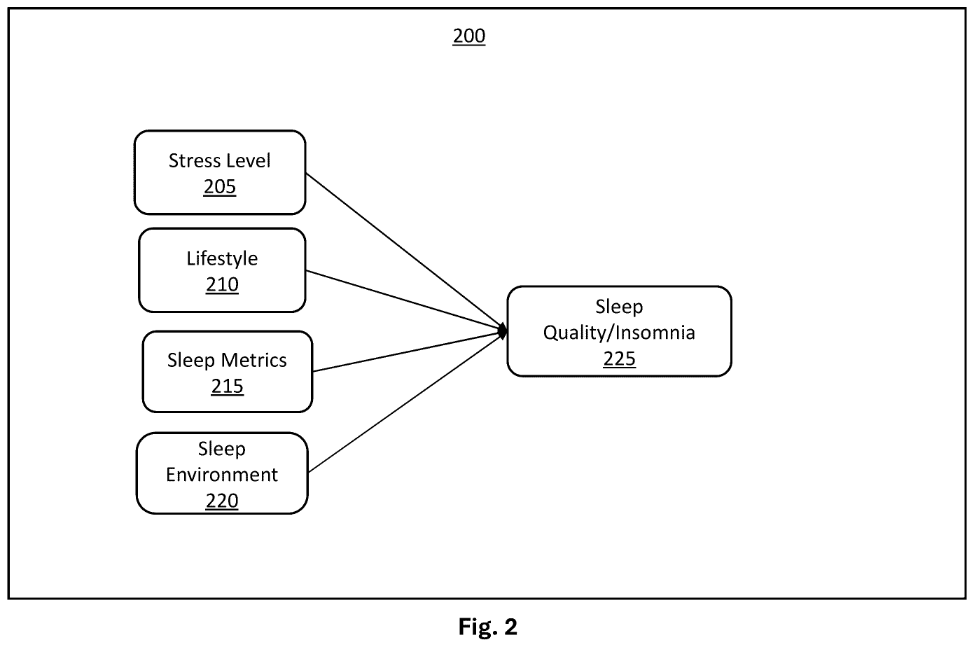

In contrast, the techniques described herein overcome these problems by using a multimodal sensing approach, using a wearable device that passively and objectively detects symptoms of sleep-related disorders and can detect these symptoms before they are recognized or appreciated by users. FIG. 1 illustrates an example method of determining an insomnia condition of a user using a wearable device that has multiple sensors that each collects information related to insomnia. The wearable device may be a watch, a pendant or necklace, a ring, a head-worn device, a chest-worn device, or any other suitable wearable device. FIG. 2 illustrates an example architecture for determining an insomnia condition of a user. The modules of the example of FIG. 2 are implemented by device 200, which may be the wearable device used to collect various sleep-related data, as described below.

Step 110 of the example method of FIG. 1 includes detecting, by each of a plurality of sensors of a wearable device worn by a user, a corresponding plurality of insomnia-related signals during at least a predetermined duration, and step 120 of the example method of FIG. 1 includes determining, for each of the plurality of insomnia-related signals, a set of insomnia-indicating signals by comparing each insomnia-related signal in that plurality of insomnia-related signals to a corresponding threshold.

For example, a wearable device may include a sensor that measures skin conductance, a galvanic skin response measured by two or more electrodes. When the user is asleep (which may be determined based on, e.g., accelerometer signals as described below), the skin-conductance sensor detects the user's skin conductance (or equivalently, skin resistance). A user's skin conductance varies as the user is awake, falls asleep, and during different sleep cycles, and therefore is correlates with sleep and sleep stages. In particular embodiments, if the user's measured skin conductance is greater than a threshold level, for example greater than 10% higher than a user's baseline skin conductance while the user is asleep, then the wearable device may generate an insomnia-indicating signal, which is passed to a trained machine-learning model, as described more fully below. For example, in this example (and in certain subsequent examples, as explained herein) the insomnia-indicating signal may include a binary indication that the user's skin conductance has exceeded a threshold. In particular embodiment, the insomnia-indicating signal may include an amount by which the user's skin conductance exceeded the threshold. In particular embodiments, the insomnia-indicating signal may include the skin-conductance signal itself as a function of time. In particular embodiments, skin conductance may be determined by stress level module 205 of device 200, as stress level module 205 processes biological data related to stress of the user.

As another example, a wearable device may include a sensor that measures a user's heart rate, such as a PPG sensor. When the user is asleep (which may be determined based on, e.g., accelerometer signals as described below), the heart-rate sensor detects the user's heart rate and, in particular embodiments, related data such as beat-to-beat intervals. In particular embodiments, if the user's measured heart rate is greater than a threshold level for a predetermined amount of time, for example greater than 20% higher than a user's baseline heart rate while the user is asleep for at least 10 minutes, then the wearable device may generate an insomnia-indicating signal, which is passed to a trained machine-learning model. As explained above, the insomnia-indicating signal may include a binary indication; an amount by which the user's heart rate exceeded the threshold; and/or the heart-rate signal itself as a function of time. In particular embodiments, the changes in heart-rate and/or beat-to-beat intervals may be determined by stress level module 205 of device 200.

In particular embodiments, if the user's measured beat-to-beat interval is less than 10% of a baseline beat-to-beat interval for the user while the user is asleep, then the wearable device may generate an insomnia-indicating signal, which is passed to a trained machine-learning model. Particular embodiments may generate an insomnia-indicating signal when the user's beat-to-beat interval and the standard deviation of the user's beat-to-beat interval are both at least 10% below corresponding baselines levels. The insomnia-indicating signal may include a binary indication; an amount by which the user's beat-to-beat interval (and related standard deviation, in particular embodiments) fell below the threshold; and/or the beat-to-beat interval signal itself as a function of time.

As another example, a wearable device may include a sensor that measures a user's exercise, such as an accelerometer. The wearable device may record the user's periods of exercise, and in particular embodiments, if the user is exercising less than a threshold amount (e.g., less than 30 minutes per day three days a week), then the wearable device may generate an insomnia-indicating signal, which is passed to a trained machine-learning model. The insomnia-indicating signal may include a binary indication; an amount by which the user's exercise is below the threshold; and/or the actual exercise durations or the accelerometer signals generated during exercise. In particular embodiments, the user's exercise data may be tracked by lifestyle module 210 of device 200, as lifestyle module processes the insomnia-related information drawn from the user's lifestyle factors.

As another example, a wearable device may track a user's screen time, which is the amount of time the user spends watching or engaging with electronic displays (e.g., a TV display, a smartphone display, etc.) In particular embodiments, screen time may be automatically tracked. For example, a wearable device may track the amount of time its screen is active each day, and allocate that time to the user's screen time for that day. As another example, the wearable device may receive screen-time information relating to another client device, such as the user's smartphone or TV. In particular embodiments, screen time information may be at least partly supplied by a user. If the user's screen time exceeds threshold amounts, then the wearable device may generate an insomnia-indicating signal. For example, if the user averages more than 6 hours of screen time each day over a particular period (e.g., a week), or if a user has used more than 30 minutes of screen time near the user's bedtime (e.g., within 1 hour of bedtime), then in each case the wearable device may generate an insomnia-indicating signal, which is passed to a trained machine-learning model. The insomnia-indicating signal may include a binary indication; an amount by which the user's screen time exceeds the threshold; and/or the actual screen-time data (e.g., the amount of screen time and the times of day in which the screen time occurred). In particular embodiments, the user's screen-time data may be tracked by lifestyle module 210 of device 200.

While the example above describes tracking screen time by a wearable device, this disclosure contemplates that screen time may be tracked by another client device or combination of client devices, such as a smartphone, TV, etc. This data may then be aggregated by a single device, such as a server device or one of the client devices, to determine the user's overall screen-time usage.

As another example, a wearable device may include an accelerometer that measures a user's movements. Accelerometer signals include characteristic signatures that correspond to sleep onset, wakefulness during periods of sleep, stages of sleep, and sleep-awakening transitions (e.g., getting up for the day), and these signatures can be based on both the magnitude and duration of the accelerometer signals. The wearable device may record the user's movements and label the corresponding periods (e.g., stages of sleep); this disclosure contemplates that the accelerometer data may be provided to another device for labeling, in particular embodiments. If the user's sleep metrics meet a threshold, then the wearable device (or the device labelling the accelerometer data) generates an insomnia-indicating signal, which is passed to a trained machine-learning model. For example, if the user is getting less than 3 hours of deep sleep less than 3 nights per week, or if the user is taking a daytime nap that is longer than 30 minutes for 3 or more days per week, then in either case an insomnia-indicating signal may be generated. Insomnia-indicating signal may include a binary indication (i.e., that the threshold was met); an amount by which the user's deep sleep is below the threshold and/or the amount by which the user's daytime naps exceed the threshold; and/or the accelerometer signals themselves. In particular embodiments, the user's sleep-related movement data may be tracked by sleep-metrics module 215 of device 200, as sleep-metrics module processes the insomnia-related information drawn from data regarding the user's sleep and sleep stages.

As another example, a wearable device may include a sensor that measures luminosity in the user's environment. For example, if the ambient light exceeds 500 lux for more than one hour while the user is asleep, then the wearable device may generate an insomnia-indicating signal, which is passed to a trained machine-learning model. Insomnia-indicating signals may include a binary indication; an amount (in lux or in temporal duration, or both) by which the ambient light exceeded the threshold; and/or the luminosity signals.

As another example, a wearable device may include a sensor (e.g., a microphone) that measures noise in the user's environment. For example, if the ambient noise exceeds 50 decibels (dB) while the user is asleep, then the wearable device may generate an insomnia-indicating signal, which is passed to a trained machine-learning model. Insomnia-indicating signals may include a binary indication; an amount by which the ambient noise exceeded the threshold; and/or the microphone signals themselves. In particular embodiments, data related to the ambient noise may be tracked by sleep-environment module 220 of device 200, as sleep-environment module processes the environmental information corresponding to the user's sleeping conditions.

As another example, a wearable device may include a sensor (e.g., a thermometer) that measures the ambient temperature and/or the temperature of the user's skin. For example, if the ambient temperature exceeds 28° C. while the user is asleep, then the wearable device may generate an insomnia-indicating signal, which is passed to a trained machine-learning model. In addition or the alternative, if the user's skin temperature increases by at least 0.5° C. relative to a baseline skin temperature while the user is asleep, then the wearable device may generate an insomnia-indicating signal. Insomnia-indicating signals may include a binary indication; an amount by which the ambient temperature and/or skin temperature exceed their respective threshold; and/or the thermometer signals themselves. In particular embodiments, sleep-related temperature data may be tracked by sleep-environment module 220 of device 200

In particular embodiments, an insomnia condition may include information related to a user's diet. For example, a user may provide data describing their diet, and this data may be provided through, e.g., an interface of the wearable device, a smartphone, a PC, etc. For example, a user may indicate each day whether they have eaten any food less than a predetermined amount of time before the user's bedtime, e.g., 60 minutes before bed. As another example, a user may indicate each day whether they have consumed alcohol or caffeine less than a predetermined amount of time before the user's bedtime, e.g., 4 hours before bed. Each condition being met may result in generating an insomnia-indicating signal, which is passed to a trained machine-learning model. In particular embodiments, the conditions are further based on a rolling threshold, e.g., if the user has eaten less than 60 minutes before bed for at least 3 days each week, then the wearable device may generate an insomnia-indicating signal. In particular embodiments, the user's diet-related data may be tracked by lifestyle module 210 of device 200.

Step 130 of the example method of FIG. 1 includes determining, by a trained machine-learning model and based on the determined sets of insomnia-indicating signals, an insomnia condition of the user. The trained machine-learning model may be deployed on the wearable device or on another device (e.g., a client device or a server device). The model may be, for example, a neural network. The model is trained on clinical data; for example, collections of training sensor signals and ground-truth insomnia conditions as diagnosed by a health professional may be input into the model to train the model to predict insomnia conditions based on the input sensor signals. The predicted insomnia condition may include a binary determination (e.g., the user has insomnia or does not have insomnia), a confidence level, a probability that the user has insomnia, and/or a grade (e.g., on a scale from 1-10) of a predicted severity of the insomnia, corresponding to the ground-truth labels used to train the machine-learning model. For example, if binary insomnia/no insomnia ground-truth classification labels are used to train the model, then the trained model will output an insomnia/no insomnia classification based on input data during an inference phase. In particular embodiments, sleep quality/insomnia module 225 of device 220 includes the trained machine-learning model, and may track and store insomnia-related information as that data is processed by and provided from specific modules 205-220.

As discussed above, the wearable device includes sensors that detect insomnia-related signals during at least a predetermined duration. For example, the predetermined duration may be two or three weeks. For instance, during the predetermined duration an accelerometer of the wearable device would detect many signals regarding the user's motion. Each of these accelerometer signals (which may segmented or windowed) is compared to one or more corresponding accelerometer thresholds, as discussed above, and thus the insomnia-indicating signals can be determined. Steps 110 and 120 are repeatedly performed during the predetermined duration, and at the end of the predetermined duration (e.g., at the end of two or three weeks), step 130 may be performed to determine the user's insomnia condition, based on the entire set of insomnia-indicating signals generated over the predetermined duration.

In particular embodiments, the wearable device or another device (e.g., a device hosting the machine-learning model) may determine whether enough data has been collected to make a prediction of the user's insomnia condition. For example, the model may require that data be collected from at least three nights a week over a predetermined duration (e.g., two-week period), although this disclosure contemplates that other data thresholds may be used. If not enough data is collected, then the user may be requested (e.g., through a UI of the wearable device) to continue monitoring for insomnia; otherwise, an insomnia prediction may be made.

As discussed above, the techniques described herein passively detect insomnia-related signals, and therefore an active assessment (e.g., an overnight stay at a clinic) is not needed to detect insomnia conditions. In addition, the techniques described herein determine a user's insomnia condition even when the user has no awareness of their condition or symptoms, and therefore can notify users of actual or potential insomnia conditions without requiring the user to actively detect their symptoms, which can be particularly challenging as many characteristics of insomnia occur while the user is asleep. In addition, a clinician who suspects a user may have insomnia can prescribe the techniques as described herein to determine whether the user has insomnia, and the techniques are based on objective evidence of the user's typical sleep-related characteristics, rather than being based on the user's subjective determinations or based on sleep characteristics in a non-typical scenario (e.g., at an overnight clinic). A user can then provide their insomnia data and determined condition to their health professional, who can use that data to aid in a formal diagnosis of insomnia or other health conditions.

In addition, the techniques described herein combine multiple sensor modalities, and therefore can detect insomnia conditions that any one sensor cannot. For example, actigraphy determines only wake vs rest; while a combination of sensors (e.g., accelerometer, PPG sensor, skin conductance, etc.) can detect many different insomnia-related conditions of the user while the user is awake, and particularly while the user is asleep. Moreover, data from multiple sensors can be synergized to provide additional insomnia-related information; for example, an accelerometer can determine whether a user is asleep, and knowing that information, a skin-conductance sensor can be activated to determine whether the user's skin conductance is changing while the user is asleep, as skin conductance is only relevant to insomnia while the user is asleep.

As illustrated in the immediately preceding example, a wearable device using multiple sensors may trigger some of those sensors for insomnia detection based on inputs such as a determination of whether the user is asleep (e.g., based on accelerometer data) or a time relative to the user's normal sleep patterns (e.g., a determination that the user is within an hour of bedtime). As a result, in particular embodiments, the multi-modality approach described herein preserves battery life by intelligently activating sensors and the corresponding software routines as conditions specific to insomnia detection occur.

In addition, as discussed above, only insomnia-indicating signals are recorded and sent to the trained machine-learning model for analysis. Thus, while the sensors may be frequently recording data, much of this data can simply be discarded if it does not meet any of the threshold described above. The techniques described herein therefore significantly save compute resources relative to techniques that would determine insomnia based directly from sensor data, e.g., relative to a techniques that record sensor data each night and send all of that data to a machine-learning model. Moreover, because only data that has passed a threshold is sent to the machine-learning model, the model receives only data that is likely to be meaningful to insomnia detection, effectively reducing the amount of noise received by the model and therefore improving detection of insomnia conditions.

In particular embodiments, a head-worn device (such as a pair of earbuds) may be worn while the user is asleep, and the head-worn device may detect an EEG signal of the user. Accelerometer data from a wearable device (e.g., the same head-worn device or a different device, such as a watch) may be used to determine whether the user is walking or stationary, and at the same time, EEG signals may be used to determine whether the user is asleep or awake. If the user is both walking and is asleep, as detected by the respective signals, the system may determine that the user is sleepwalking. Sleepwalking may, in addition or the alternative, be detected by comparing a user's gait during waking walking to a user's detected gait, as gait abnormalities may indicate sleepwalking.

The disclosure at times refers to a “day” or to a user's “bedtime,” and this disclosure recognizes that these terms may vary from person to person. For example, for a person who works on a night shift, that person's “day” may correspond to different hours than a person who works an 8 am-5 pm job. Likewise, different users have different nighttime schedules and routines, and therefore can have different bedtimes. A user's daytime or bedtime may be determined based on, for example, a user's input, and/or maybe automatically detected, e.g., based on the user's detected activity, and the determined periods of “daytime” and “bedtime” may then be used for that user. In other words, because certain signals are collected during the user's day, around the user's bedtime, and during the user's nighttime, this signal collection and sensor activation may be personalized to each user's individual routines and circumstances.

In particular embodiments, a user's stress level may be estimated and used by a trained machine-learning model to estimate a user's insomnia condition. For example, galvanic skin response along with heart-rate variability and bran waves may be used to estimate stress and/or sleep arousal.

In particular embodiments, an insomnia condition may be used to estimate other health conditions of a user, perhaps along with sensor signals obtained during insomnia detection. For example, insomnia is related to dementia and other neurological disorders, and therefore insomnia may be used as an indicator for such conditions (along with, for example, data regarding a user's gait).

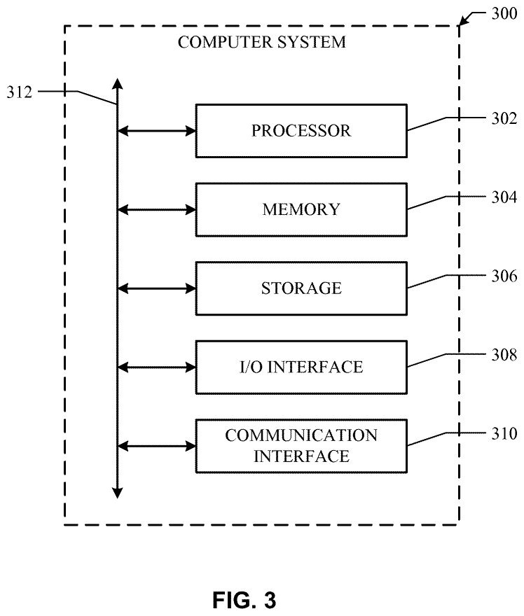

FIG. 3 illustrates an example computer system 300. In particular embodiments, one or more computer systems 300 perform one or more steps of one or more methods described or illustrated herein. In particular embodiments, one or more computer systems 300 provide functionality described or illustrated herein. In particular embodiments, software running on one or more computer systems 300 performs one or more steps of one or more methods described or illustrated herein or provides functionality described or illustrated herein. Particular embodiments include one or more portions of one or more computer systems 300. Herein, reference to a computer system may encompass a computing device, and vice versa, where appropriate. Moreover, reference to a computer system may encompass one or more computer systems, where appropriate.

This disclosure contemplates any suitable number of computer systems 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 300 may include one or more computer systems 300; be unitary or distributed; span multiple locations; span multiple machines; span multiple data centers; or reside in a cloud, which may include one or more cloud components in one or more networks. Where appropriate, one or more computer systems 300 may perform without substantial spatial or temporal limitation one or more steps of one or more methods described or illustrated herein. As an example and not by way of limitation, one or more computer systems 300 may perform in real time or in batch mode one or more steps of one or more methods described or illustrated herein. One or more computer systems 300 may perform at different times or at different locations one or more steps of one or more methods described or illustrated herein, where appropriate.

In particular embodiments, computer system 300 includes a processor 302, memory 304, storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 302 includes hardware for executing instructions, such as those making up a computer program. As an example and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306. In particular embodiments, processor 302 may include one or more internal caches for data, instructions, or addresses. This disclosure contemplates processor 302 including any suitable number of any suitable internal caches, where appropriate. As an example and not by way of limitation, processor 302 may include one or more instruction caches, one or more data caches, and one or more translation lookaside buffers (TLBs). Instructions in the instruction caches may be copies of instructions in memory 304 or storage 306, and the instruction caches may speed up retrieval of those instructions by processor 302. Data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches may speed up read or write operations by processor 302. The TLBs may speed up virtual-address translation for processor 302. In particular embodiments, processor 302 may include one or more internal registers for data, instructions, or addresses. This disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In particular embodiments, memory 304 includes main memory for storing instructions for processor 302 to execute or data for processor 302 to operate on. As an example and not by way of limitation, computer system 300 may load instructions from storage 306 or another source (such as, for example, another computer system 300) to memory 304. Processor 302 may then load the instructions from memory 304 to an internal register or internal cache. To execute the instructions, processor 302 may retrieve the instructions from the internal register or internal cache and decode them. During or after execution of the instructions, processor 302 may write one or more results (which may be intermediate or final results) to the internal register or internal cache. Processor 302 may then write one or more of those results to memory 304. In particular embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which may each include an address bus and a data bus) may couple processor 302 to memory 304. Bus 312 may include one or more memory buses, as described below. In particular embodiments, one or more memory management units (MMUs) reside between processor 302 and memory 304 and facilitate accesses to memory 304 requested by processor 302. In particular embodiments, memory 304 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 304 may include one or more memories 304, where appropriate. Although this disclosure describes and illustrates particular memory, this disclosure contemplates any suitable memory.

In particular embodiments, storage 306 includes mass storage for data or instructions. As an example and not by way of limitation, storage 306 may include a hard disk drive (HDD), a floppy disk drive, flash memory, an optical disc, a magneto-optical disc, magnetic tape, or a Universal Serial Bus (USB) drive or a combination of two or more of these. Storage 306 may include removable or non-removable (or fixed) media, where appropriate. Storage 306 may be internal or external to computer system 300, where appropriate. In particular embodiments, storage 306 is non-volatile, solid-state memory. In particular embodiments, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 may include one or more storage control units facilitating communication between processor 302 and storage 306, where appropriate. Where appropriate, storage 306 may include one or more storages 306. Although this disclosure describes and illustrates particular storage, this disclosure contemplates any suitable storage.

In particular embodiments, I/O interface 308 includes hardware, software, or both, providing one or more interfaces for communication between computer system 300 and one or more I/O devices. Computer system 300 may include one or more of these I/O devices, where appropriate. One or more of these I/O devices may enable communication between a person and computer system 300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 308 for them. Where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In particular embodiments, communication interface 310 includes hardware, software, or both providing one or more interfaces for communication (such as, for example, packet-based communication) between computer system 300 and one or more other computer systems 300 or one or more networks. As an example and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it. As an example and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 300 may include any suitable communication interface 310 for any of these networks, where appropriate. Communication interface 310 may include one or more communication interfaces 310, where appropriate. Although this disclosure describes and illustrates a particular communication interface, this disclosure contemplates any suitable communication interface.

In particular embodiments, bus 312 includes hardware, software, or both coupling components of computer system 300 to each other. As an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 312 may include one or more buses 312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
detecting, by each of a plurality of sensors of a wearable device worn by a user, a corresponding plurality of insomnia-related signals during at least a predetermined duration;
determining, for each of the plurality of insomnia-related signals, a set of insomnia-indicating signals by comparing each insomnia-related signal in that plurality of insomnia-related signals to a corresponding threshold; and
determining, by a trained machine-learning model and based on the determined sets of insomnia-indicating signals, an insomnia condition of the user.

2. The method of claim 1, wherein the wearable device comprises a watch.

3. The method of claim 1, wherein:
one of the plurality of insomnia-related signals comprises a skin conductance of the user while the user is asleep; and
the corresponding threshold comprises a 10% increase relative to a baseline skin conductance of the user while the user is asleep.

4. The method of claim 1, wherein one or more of:
(1) one of the plurality of insomnia-related signals comprises a heart rate of the user while the user is asleep; and
the corresponding threshold comprises a 20% increase, for at least a predetermined period of time, relative to a baseline heart rate of the user while the user is asleep; or
(2) one of the plurality of insomnia-related signals comprises a beat-to-beat interval of the user while the user is asleep; and
the corresponding threshold comprises a 10% decrease relative to a baseline beat-to-beat interval of the user while the user is asleep.

5. The method of claim 1, wherein:
one of the plurality of insomnia-related signals comprises a duration of exercise; and
the corresponding threshold comprises 30 minutes per day for a threshold number of days per week.

6. The method of claim 1, wherein one or more of:
(1) one of the plurality of insomnia-related signals comprises an amount of average screen time per day; and
the corresponding threshold comprises 6 hours; or
(2) one of the plurality of insomnia-related signals comprises an amount of screen time before the user's bedtime; and
the corresponding threshold comprises 30 minutes.

7. The method of claim 1, wherein one or more of:
(1) one of the plurality of insomnia-related signals comprises an amount of deep sleep; and
the corresponding threshold comprises 3 hours for at least a threshold number of nights each week; or
(2) one of the plurality of insomnia-related signals comprises a nap duration during the user's waking hours; and
the corresponding threshold comprises 30 minutes per day for a least a threshold number of days per week.

8. The method of claim 1, wherein:
one of the plurality of insomnia-related signals comprises an amount of ambient light while the user is asleep; and
the corresponding threshold comprises 500 lux for at least one hour.

9. The method of claim 1, wherein one or more of:
(1) one of the plurality of insomnia-related signals comprises a skin temperature while the user is asleep; and
the corresponding threshold comprises a 0.5° C. increase relative to a baseline skin temperature of the user while the user is asleep; or
(2) one of the plurality of insomnia-related signals comprises an ambient air temperature while the user is asleep; and
the corresponding threshold comprises 28° C.

10. The method of claim 1, wherein:
one of the plurality of insomnia-related signals comprises an ambient noise level while the user is asleep; and
the corresponding threshold comprises 50 db.

11. The method of claim 1, further comprising:
receiving, from the user, a description of the user's food intake, alcohol intake, or caffeine intake before the user's bedtime; and
further determining the insomnia condition of the user, by the trained machine-learning model, based on the description.

12. The method of claim 1, wherein the predetermined duration comprises two weeks.

13. The method of claim 1, further comprising determining whether the user is sleepwalking by:
determining, based on a signal obtained by an accelerometer of the wearable device, whether the user is walking; and
determining, by an EEG signal from a head-worn device of the user, whether the user is asleep.

14. A wearable device comprising:
a plurality of sensors, each sensor configured to detect a corresponding plurality of insomnia-related signals during at least a predetermined duration while the wearable device is worn by the user; and
one or more non-transitory computer readable storage media storing instructions; and one or more processors coupled to the one or more non-transitory computer readable storage media and operable to execute the instructions to:
determine, for each of the plurality of insomnia-related signals, a set of insomnia-indicating signals by comparing each insomnia-related signal in that plurality of insomnia-related signals to a corresponding threshold; and
determine, by a trained machine-learning model and based on the determined sets of insomnia-indicating signals, an insomnia condition of the user.

15. The system of claim 14, wherein the wearable device comprises a watch.

16. The system of claim 14, wherein:
one of the plurality of insomnia-related signals comprises a skin conductance of the user while the user is asleep; and
the corresponding threshold comprises a 10% increase relative to a baseline skin conductance of the user while the user is asleep.

17. The system of claim 14, wherein one or more of:
(1) one of the plurality of insomnia-related signals comprises a heart rate of the user while the user is asleep; and
the corresponding threshold comprises a 20% increase, for at least a predetermined period of time, relative to a baseline heart rate of the user while the user is asleep; or
(2) one of the plurality of insomnia-related signals comprises a beat-to-beat interval of the user while the user is asleep; and
the corresponding threshold comprises a 10% decrease relative to a baseline beat-to-beat interval of the user while the user is asleep.

18. The system of claim 14, wherein one or more of:
(1) one of the plurality of insomnia-related signals comprises an amount of deep sleep; and
the corresponding threshold comprises 3 hours for at least a threshold number of nights each week; or
(2) one of the plurality of insomnia-related signals comprises a nap duration during the user's waking hours; and
the corresponding threshold comprises 30 minutes per day for a least a threshold number of days per week.

19. The system of claim 14, wherein the predetermined duration comprises two weeks.

20. One or more non-transitory computer readable storage media storing instructions that are operable when executed to:
access, from each of a plurality of sensors of a wearable device worn by a user, a corresponding plurality of insomnia-related signals during at least a predetermined duration;
determine, for each of the plurality of insomnia-related signals, a set of insomnia-indicating signals by comparing each insomnia-related signal in that plurality of insomnia-related signals to a corresponding threshold; and
determine, by a trained machine-learning model and based on the determined sets of insomnia-indicating signals, an insomnia condition of the user.
